(19) United States
(12) Patent Application Publication
(10) Pub. No.: US 2010/0166813 A1
(43) Pub. Date: Jul. 1, 2010

(54) HAIR TREATMENT COMPOSITIONS COMPRISING PARTICLES AND HYDROPHOBIC OIL

(75) Inventors: Caroline Alexandra HALL, Wirral (GB); Paul David JENKINS, Wirral (GB)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO INC., D/B/A UNILEVER, Englewood Cliffs, NJ (US)

(21) Appl. No.: 12/623,745

(22) Filed: Nov. 23, 2009

Related U.S. Application Data
(62) Division of application No. 11/883,432, filed on Mar. 11, 2008, now abandoned.

(30) Foreign Application Priority Data
Feb. 1, 2005 (EP) 05250546

(51) Int. Cl. A61K 8/92; A61K 8/02; A61Q 5/02; A61Q 5/06

(52) U.S. Cl. 424/401; 424/70.1; 424/70.11; 424/70.21; 424/70.22; 424/70.27; 424/70.31

(57) ABSTRACT

A hair treatment composition comprising studded particles, the studded particles comprising liquid hydrophobic oil droplets studded by particulate material, the mean diameter (D3.2) of the studded particles being from 0.01 to 100 microns, and hair treatment composition obtainable by the process of: (i) forming a pre-mix of a dispersion of studded particles, the studded particles comprising liquid hydrophobic oil studded by particulate material; the mean diameter (D3,2) of the studded particles being from 0.01 to 100 microns (ii) forming a further base formulation (iii) adding the resulting dispersion to the base formulation; and in which the pre-mix comprises less than 0.5 wt % of free surfactant.

This is a divisional application of co-pending U.S. patent application Ser. No. 11/883,432 (entitled “Hair Treatment Compositions Comprising Particles and Hydrophobic Oil”) filed on Mar. 11, 2008, which is a national stage application under 35 U.S.C. §371 of PCT International Application PCT/EP2006/000089 filed Jan. 9, 2006, which claims priority under 35 U.S.C. §119 to EP Application No. 05250546 filed Feb. 1, 2005; all of which are incorporated herein, in their entirety, by reference.

### FIELD OF THE INVENTION

The invention relates to hair treatment compositions. More particularly the invention relates to compositions that give increased stylability and volume to the hair. The compositions of the invention have also been found to condition the hair.

### BACKGROUND AND PRIOR ART

Consumers require hair that can be easily styled, yet feel well conditioned. With conventional hair styling products there is normally a trade-off between styling and conditioning; in that hair that is well conditioned normally proves difficult to style.

Particulate material are known to add body to the hair. WO01/30310 describes hair treatment compositions comprising an unagglomerated particulate substance; the composition is said to give the composition body and to aid styling.

The present invention provides compositions that can be used to style and increase the volume of hair, yet leave the hair feeling well conditioned.

In a first aspect the present invention relates to a hair treatment composition comprising studded particles, the studded particles comprising liquid hydrophobic oil droplets studded by particulate material, the mean diameter (D3.2) of the studded particles being from 0.01 to 100 microns.

### BRIEF DESCRIPTION OF DRAWINGS

FIG. 1 is a side-by-side presentation of photomicrographs at different magnifications of a hair section that has been immersed in the formulation of Example D.

FIG. 2 is a side-by-side presentation of photomicrographs of a hair section that has been immersed in the formulation of Example 10.

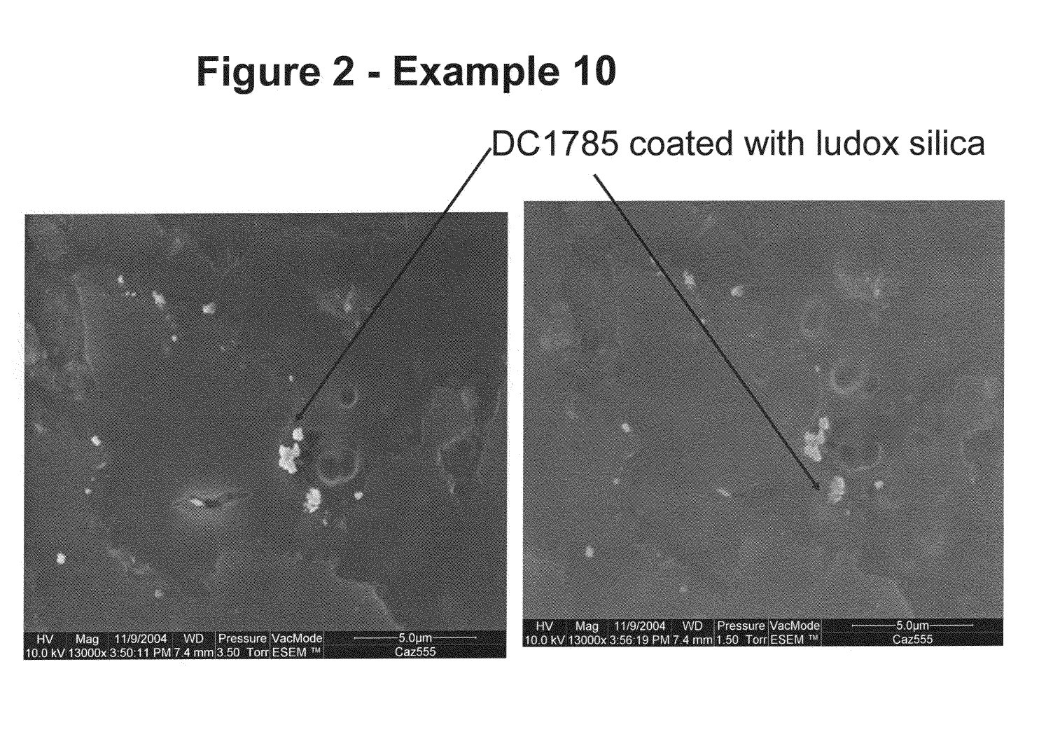

### DESCRIPTION OF THE INVENTION

In a second aspect the invention provides a hair treatment composition obtainable by the process of:
i) forming a pre-mix of a dispersion of studded particles, the studded particles comprise liquid hydrophobic oil studded by particulate material; the mean diameter (D3,2) of the studded particles being from 0.01 to 100 microns;
ii) forming a base composition
iii) adding the resulting dispersion to the base composition; and
   in which the pre-mix comprises less than 0.5 wt % of free surfactant.

The invention also relates to a method for enhancing the friction/adhesion between hair fibres by applying a composition as described above.

A further aspect of the invention is the use of the above composition for increasing the volume of hair

The invention also describes a process for preparing a hair treatment composition comprising the steps of:
i) forming a pre-mix of a dispersion of studded particles, the studded particles comprising liquid hydrophobic oil studded by particulate material; the mean diameter (D3,2) of the studded particles being from 0.01 to 100 microns
ii) adding the resulting dispersion to a base composition;
   in which the pre-mix comprises less than 0.5 wt % of free surfactant.

### DETAILED DESCRIPTION

### The Premix

Compositions of the invention comprise the formation of a pre-mix. The pre-mix is a dispersion of studded particles. The studded particles comprise liquid hydrophobic oil studded by particulate material. The mean diameter (D3,2) of the studded particles is from 0:01 to 100 microns, more preferably from 0.4 microns to 30 microns.

Preferably the dispersion is in an aqueous base.

The pre-mix comprises less than 0.5 wt % of free surfactant. In the context of the present invention the term free surfactant means surfactant that does not have an emulsifying role; that is the surfactant is not present at an oil-water interface but is free in solution.

It is preferable if the level of emulsifying surfactant within the pre-mix is below 7 wt %, more preferably below 3.5 wt %, most preferably below 2 wt %. In a preferred process for producing the pre-mix the liquid hydrophobic oil is added to the water to form a dispersion followed by addition of the particulate material.

It is advantageous if within the premix the weight ratio of studded particles to solvent is 1:1 or less, preferably 1:2 or less.

The weight ratio of hydrophobic oil to particulate material within the studded particulate is preferably within the range 20:1 to 1;40, more preferably 10:1 to 1;25.

### The Hydrophobic Oil

It is preferable if the liquid hydrophobic oil material has a particle size of 0.01 to 100 microns more preferably 0.03 micron to 20 microns

In most instances the hydrophobic oil is water-insoluble. By “insoluble” is meant that the material is not soluble in water (distilled or equivalent) at a concentration of 0.1% (w/w), at 25° C.

It is preferable if the hydrophobic oil is a silicone conditioning agent.

Suitable silicones include polydiorganosiloxanes, in particular polydimethylsiloxanes that have the CTFA designation dimethicone. Also suitable for use compositions of the invention (particularly shampoos and conditioners) are polydimethyl siloxanes having hydroxyl end groups, which have the CTFA designation dimethiconol. Also suitable for use in compositions of the invention are silicone gums having a slight degree of cross-linking, as are described for example in WO 96/31188.

The viscosity of the emulsified silicone itself (not the emulsion or the final hair conditioning composition) is usually at least 100 cst at 25° C., preferably at least 1,000 cst, more preferably at least 10,000, most preferably at least 50,000 cst. Preferably the viscosity does not exceed 10⁹ cst for ease of formulation.

In can be advantageous for ease of manufacture if the silicones are added to the pre-mix as pre-formed emulsions. Examples of suitable pre-formed emulsions include emulsions DC1310, D2-1766, DC2-1784, DC-1785, DC-1786, DC-1788 and microemulsions DC2-1865 and DC2-1870, all available from Dow Corning. These are all emulsions/microemulsions of dimethiconol. Cross-linked silicone gums are also available in a pre-emulsified form, which is advantageous for ease of formulation, e.g. DC-1787, DC-2220, DC-9509.

A further preferred class of silicones for inclusion in shampoos and conditioners of the invention are amino functional silicones. Examples of suitable amino functional silicones include: polysiloxanes having the CTFA designation “aminodimethicone”.

Specific examples of amino functional silicones suitable for use in the invention are the aminosilicone oils DC2-8220, DC2-8166, DC2-8466, and DC2-8950-114 (all ex Dow Corning), and GE 1149-75, (ex General Electric Silicones). Suitable quaternary silicone polymers are described in EP-A-0 530 974. A preferred quaternary silicone polymer is K3474, ex Goldschmidt.

Also suitable are emulsions of amino functional silicone oils with non-ionic and/or cationic surfactant.

Pre-formed emulsions of amino functional silicone are also available from suppliers of silicone oils such as Dow Corning and General Electric. Specific examples include DC929 Cationic Emulsion, DC939 Cationic Emulsion, and the non-ionic emulsions DC2-7224, DC2-8467, DC2-8177 and DC2-8154 (all ex Dow Corning).

Further examples of hydrophobic oils include hydrocarbon oils, fatty esters and mixtures thereof. Straight chain hydrocarbon oils will preferably contain from about 12 to about 30 carbon atoms. Also suitable are polymeric hydrocarbons of alkenyl monomers, such as C₂-C_{E}, alkenyl monomers.

Specific examples of suitable hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, and mixtures thereof. Branched-chain isomers of these compounds, as well as of higher chain length hydrocarbons, can also be used.

Suitable fatty esters are characterised by having at least 10 carbon atoms, and include esters with hydrocarbyl chains derived from fatty acids or alcohols, Monocarboxylic acid esters include esters of alcohols and/or acids of the formula R′COOR in which R′ and R independently denote alkyl or alkenyl radicals and the sum of carbon atoms in R′ and R is at least 10, preferably at least 20. Di- and trialkyl and alkenyl esters of carboxylic acids can also be used.

Particularly preferred fatty esters are mono-, di- and triglycerides, more specifically the mono-, di-, and tri-esters of glycerol and long chain carboxylic acids such as C₁-C₂₂ carboxylic acids. Preferred materials include cocoa butter, palm stearin, sunflower oil, soyabean oil and coconut oil.

### The Particulate Material

The pre-mix of the invention is formed from a dispersion of studded particles. The particulate material that forms the studs of the particles can be any suitable particulate.

The particulate material may contain metals such as silicon, aluminium, titanium, zinc, gold, silver, iron, zinc, copper and combinations thereof. The metals may possess a zero oxidation state, e.g gold, silver, or be in oxidised or reduced form, e.g as in silica, alumina, titania, zinc oxide, iron oxide, zinc sulfide.

A preferred form of particulate material is surface modified silica, especially useful is silica surface modified with aluminium chloride (sold under the trade name Ludox CL ex Grace Davison.)

Examples of suitable particles are those described in Matijević E., Chem. Mater., 1993, vol. 5, pp. 412-426 and Matijević E., Langmuir, 1994, vol. 10, pp. 8-16).

Particulates with an aspect ratio greater than 5:1 are not preferred.

It is preferable if the particulate has a mean diameter (D3,2) less than 500 nm, more preferably less than 250 nm most preferably less than 100 nm.

Particles may also be composed of organic monomers reacted so to form spherical 3D polymer networks.

It is advantageous if the particulate material does not have any coating.

### Product Form

The final product form of hair treatment compositions according to the invention may suitably be, for example, shampoos, conditioners, sprays, mousses, gels, waxes or lotions.

Preferred product forms rinse off products, particularly shampoos and post-wash conditioners

The pH of the formulations of the invention are in the range from pH 3 to pH 11, more preferably used at a pH from 3 to 8.

### Hair Treatment Composition Base Formulation

Shampoo compositions preferably comprise one or more cleansing surfactants, which are cosmetically acceptable and suitable for topical application to the hair. Further surfactants may be present as emulsifiers.

Suitable cleansing surfactants, are selected from anionic, amphoteric and zwitterionic surfactants, and mixtures thereof.

The cleansing surfactant may be the same surfactant as the emulsifier, or may be different.

### Anionic Cleansing Surfactant

Shampoo compositions according to the invention will typically comprise one or more anionic cleansing surfactants which are cosmetically acceptable and suitable for topical application to the hair.

Examples of suitable anionic cleansing surfactants are the alkyl sulphates, alkyl ether sulphates, alkaryl sulphonates, alkanoyl isethionates, alkyl succinates, alkyl sulphosuccinates, N-alkyl sarcosinates, alkyl phosphates, alkyl ether phosphates, alkyl ether carboxylates, and alpha-olefin sulphonates, especially their sodium, magnesium, ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from 1 to 10 ethylene oxide or propylene oxide units per molecule.

Typical anionic cleansing surfactants for use in shampoo compositions of the invention include sodium oleyl sulpho succinate, ammonium lauryl sulphosuccinate, ammonium lauryl sulphate, sodium cocoyl isethionate, sodium lauryl isethionate and sodium N-lauryl sarcosinate. The most preferred anionic surfactants are sodium lauryl sulphate, sodium lauryl ether sulphate(n)EO, (where n ranges from 1 to 3), ammonium lauryl sulphate and ammonium lauryl ether sulphate(n)EO, (where n ranges from 1 to 3).

The total amount, of anionic cleansing surfactant in shampoo compositions of the invention is generally from 5 to 30, preferably from 6 to 20, more preferably from 8 to 16 wt % of the total composition.

### Co-Surfactant

The shampoo composition can optionally include co-surfactants, preferably an amphoteric or zwitterionic surfactant, which can be included in an amount ranging from 0 to about 8, preferably from 1 to 4 wt %.

Examples of amphoteric and zwitterionic surfactants include, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine and preferably lauryl betaine, cocamidopropyl betaine and sodium cocamphopropionate.

Another preferred co-surfactant is a nonionic surfactant, which can be included in an amount ranging from 0 to 8 wt %, preferably from 2 to 5 wt % of the total composition.

For example, representative nonionic surfactants that can be included in shampoo compositions of the invention include condensation products of aliphatic (C₈-C₁₈) primary or secondary linear or branched chain alcohols or phenols with alkylene oxides, usually ethylene oxide and generally having from 6 to 30 ethylene oxide groups.

Further nonionic surfactants which can be included in shampoo compositions of the invention are the alkyl polyglycosides (APGs). Typically, the APG is one which comprises an alkyl group connected (optionally via a bridging group) to a block of one or more glycosyl groups. Preferred APGs are defined by the following formula:

RO-(G)ₙ

wherein R is a branched or straight chain C₅ to C₂₀ alkyl or alkenyl group, G is a saccharide group and n is from 1 to 10.

Other sugar-derived nonionic surfactants which can be included in shampoo compositions of the invention include the C₁₀-C₁₈ N-alkyl (C₁-C₆) polyhydroxy fatty acid amides, such as the C₁₂-C₁₆ N-methyl glucamides, as described for example in WO 92 06154 and U.S. Pat. No. 5,194,639, and the N-alkoxy polyhydroxy fatty acid amides, such as C₁₀-C₁₈ N-(3-methoxypropyl) glucamide.

The shampoo composition can also optionally include one or more cationic co-surfactants included in an amount ranging from 0.01 to 10, more preferably from 0.05 to 5, most preferably from 0.05 to 2 wt % of the total composition. Useful cationic surfactants are described herein in relation to conditioner compositions.

The total amount of surfactant (including any co-surfactant, and/or any emulsifier) in shampoo compositions of the invention is generally from 5 to 50, preferably from 5 to 30, more preferably from 10 to 25 wt %.

### Cationic Deposition Polymer

A cationic polymer is a preferred ingredient, especially in shampoo compositions of the invention.

The cationic polymer may be a homopolymer or be formed from two or more types of monomers. The molecular weight of the polymer will generally be between 5 000 and 10 000 000 Dalton, typically at least 10 000 and preferably from 100 000 to 2 000 000. The polymers will have cationic nitrogen containing groups such as quaternary ammonium or protonated amino groups, or a mixture thereof.

The cationic nitrogen-containing group will generally be present as a substituent on a fraction of the total monomer units of the cationic polymer. Thus when the polymer is not a homopolymer it can contain spacer non-cationic monomer units. Such polymers are described in the CTFA Cosmetic Ingredient Directory, 3rd edition. The ratio of the cationic to non-cationic monomer units is selected to give a polymer having a cationic charge density in the required range.

Suitable cationic deposition polymers include, for example, copolymers of vinyl monomers having cationic amine or quaternary ammonium functionalities with water soluble spacer monomers such as (meth)acrylamide, alkyl and dialkyl (meth)acrylamides, alkyl(meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition. In general secondary and tertiary amines, especially tertiary, are preferred.

Amine substituted vinyl monomers and amines can be polymerized in the amine form and then converted to ammonium by quaternization.

The cationic deposition polymers can comprise mixtures of monomer units derived from amine- and/or quaternary ammonium-substituted monomer and/or compatible spacer monomers.

Suitable cationic deposition polymers include, for example:
copolymers of 1-vinyl-2-pyrrolidine and 1-vinyl-3-methyl-imidazolium salt (e.g. chloride salt), referred to in the industry by the Cosmetic, Toiletry, and Fragrance Association, (CTFA) as Polyquaternium-16. This material is commercially available from BASF Wyandotte Corp. (Parsippany, N.J., USA) under the LUVIQUAT tradename (e.g. LUVIQUAT FC 370);
copolymers of 1-vinyl-2-pyrrolidine and dimethylaminoethyl methacrylate, referred to in the industry (CTFA) as Polyquaternium-11. This material is available commercially from Gaf Corporation (Wayne, N.J., USA) under the GAFQUAT tradename (e.g., GAFQUAT 755N);
cationic diallyl quaternary ammonium-containing polymers including, for example, dimethyldiallyammonium chloride homopolymer and copolymers of acrylamide and dimethyldiallylammonium chloride, referred to in the industry (CTFA) as Polyquaternium 6 and Polyquaternium 7, respectively;
mineral acid salts of amino-alkyl esters of homo- and co-polymers of unsaturated carboxylic acids having from 3 to 5 carbon atoms, (as described in U.S. Pat. No. 4,009,256);
cationic polyacrylamides(as described in WO95/22311).

Other cationic deposition polymers that can be used include cationic polysaccharide polymers, such as cationic cellulose derivatives, cationic starch derivatives, and cationic guar gum derivatives. Suitably, such cationic polysaccharide polymers have a charge density from 0.1 to 4 meq/g.

Cationic polysaccharide polymers suitable for use in compositions of the invention include those of the formula:

A-O—[R—N′(R¹)(R²)(R³)X-],

wherein: A is an anhydroglucose residual group, such as a starch or cellulose anhydroglucose residual. R is an alkylene, oxyalkylene, polyoxyalkylene, or hydroxyalkylene group, or combination thereof. R¹, R² and R³ independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R¹, R² and R³) is preferably about 20 or less, and X is an anionic counterion.

Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

Other suitable cationic polysaccharide polymers include quaternary nitrogen-containing cellulose ethers (e.g. as described in U.S. Pat. No. 3,962,418), and copolymers of etherified cellulose and starch (e.g. as described in U.S. Pat. No. 3,958,581).

A particularly suitable type of cationic polysaccharide polymer that can be used is a cationic guar gum derivative, such as guar hydroxypropyltrimonium chloride (commercially available from Rhone-Poulenc in their JAGUAR trademark series).

Examples are JAGUAR C13S, which has a low degree of substitution of the cationic groups and high viscosity. JAGUAR C15, having a moderate degree of substitution and a low viscosity, JAGUAR C17 (high degree of substitution, high viscosity), JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution.

Preferably the cationic deposition polymer is selected from cationic cellulose and cationic guar derivatives. Particularly preferred cationic polymers are JAGUAR C13S, JAGUAR C15, JAGUAR C17 and JAGUAR C16 and JAGUAR C162.

The cationic deposition polymer will generally be present in compositions of the invention at levels of from 0.01 to 5, preferably from 0.02 to 1, more preferably from 0.04 to 0.5 percent by weight of the composition.

### Conditioning Surfactant

Conditioner compositions usually comprise one or more conditioning surfactants which are cosmetically acceptable and suitable for topical application to the hair. Suitable conditioning surfactants are selected from cationic surfactants, used singly or in a mixture.

Cationic surfactants useful in compositions of the invention contain amino or quaternary ammonium hydrophilic moieties which are positively charged when dissolved in the aqueous composition of the present invention.

Examples of suitable cationic surfactants are those corresponding to the general formula:

[N(R₁)(R₂)(R₃)(R₄)]⁺_{(X)}⁻

in which R₁, R₂, R₃, and R₄ are independently selected from (a) an aliphatic group of from 1 to 22 carbon atoms, or (b) an aromatic, alkoxy, polyoxyalkylene, alkylamido, hydroxyalkyl, aryl or alkylaryl group having up to 22 carbon atoms; and X is a salt-forming anion such as those selected from halogen, (e.g. chloride, bromide), acetate, citrate, lactate, glycolate, phosphate nitrate, sulphate, and alkylsulphate radicals.

The aliphatic groups can contain, in addition to carbon and hydrogen atoms, ether linkages, and other groups such as amino groups. The longer chain aliphatic groups, e.g., those of about 12 carbons, or higher, can be saturated or unsaturated.

The most preferred cationic surfactants for conditioner compositions of the present invention are monoalkyl quaternary ammonium compounds in which the alkyl chain length is C16 to C22.

Examples of suitable cationic surfactants include quaternary ammonium compounds, particularly trimethyl quaternary compounds.

Preferred quaternary ammonium compounds include cetyltrimethylammonium chloride, benzyltrimethylammonium chloride (BTAC), cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, PEG-2 oleylammonium chloride and salts of these where the chloride is replaced by halogen (e.g., bromide), acetate, citrate, lactate, glycolate, phosphate nitrate, sulphate, or alkylsulphate. Further suitable cationic surfactants include those materials having the CTFA designations Quaternium-5, Quaternium-31 and Quaternium-18. Mixtures of any of the foregoing materials may also be suitable. A particularly useful cationic surfactant for use in hair conditioners of the invention is cetyltrimethylammonium chloride, available commercially, for example as GENAMIN CTAC, ex Hoechst Celanese.

Salts of primary, secondary, and tertiary fatty amines are also suitable cationic surfactants. The alkyl groups of such amines preferably have from 12 to 22 carbon atoms, and can be substituted or unsubstituted.

Particularly useful are amido substituted tertiary fatty amines, in particular tertiary amines having one C₁₂ to C₂₂ alkyl or alkenyl chain. Such amines, useful herein, include stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, rachidamidopropyldimethylamine, arachid amidopropyldiethylamine, rachidamidoethyldiethylamine, arachidamidoethyldimethylamine, diethylaminoethylstearamide. Also useful are dimethylstearamine, dimethylsoyamine, soyamine, myristylamine, tridecylamine, ethylstearylamine, N-tallowpropane diamine, ethoxylated (with 5 moles of ethylene oxide) stearylamine, dihydroxyethylstearylamine, and arachidyl behenylamine.

These amines are typically used in combination with an acid to provide the cationic species. The preferred acid useful herein includes L-glutamic acid, lactic acid, hydrochloric acid, malic acid, succinic acid, acetic acid, fumaric acid, tartaric acid, citric acid, L-glutamic hydrochloride, and mixtures thereof; more preferably L-glutamic acid, lactic acid, citric acid. Cationic amine surfactants included among those useful in the present invention are disclosed in U.S. Pat. No. 4,275,055 to Nachtigal, et al., issued Jun. 23, 1981.

The molar ratio of protonatable amines to H′ from the acid is preferably from about 1:0.3 to 1:1.2, and more preferably from about 1:0.5 to about 1:1.1.

In the conditioners of the invention, the level of cationic surfactant is preferably from 0.01 to 10 wt % of the total composition, more preferably 0.05 to 5 wt % of the total composition.

The cationic surfactants detailed in this section are also suitable for use in the aspect of the invention wherein a cationic surfactant is intimately mixed with the thermotropic mesogenic material and with oily conditioning material prior to the incorporation of the conditioning material into the final hair conditioning composition

### Fatty Materials

Conditioner compositions of the invention preferably additionally comprise fatty materials. The combined use of fatty materials and cationic surfactants in conditioning compositions is believed to be especially advantageous, because this leads to the formation of a structured phase, in which the cationic surfactant is dispersed.

By “fatty material” is meant a fatty alcohol, an alkoxylated fatty alcohol, a fatty acid or a mixture thereof.

Preferably, the alkyl chain of the fatty material is fully saturated.

Representative fatty materials comprise from 8 to 22 carbon atoms, more preferably 16 to 22. Examples of suitable fatty alcohols include cetyl alcohol, stearyl alcohol and mixtures thereof. The use of these materials is also advantageous in that they contribute to the overall conditioning properties of compositions of the invention.

Alkoxylated, (e.g. ethoxylated or propoxylated) fatty alcohols having from about 12 to about 18 carbon atoms in the alkyl chain can be used in place of, or in addition to, the fatty alcohols themselves. Suitable examples include ethylene glycol cetyl ether, polyoxyethylene (2) stearyl ether, polyoxyethylene (4) cetyl ether, and mixtures thereof.

The level of fatty alcohol material in conditioners of the invention is suitably from 0.01 to 15 wt %, preferably from 0.1 to 10 wt % of the total composition. The weight ratio of cationic surfactant to fatty alcohol is suitably from 10:1 to 1:10, preferably from 4:1 to 1:8, optimally from 1:1 to 1:7.

### Suspending Agents

In a preferred embodiment, the shampoo compositions of this invention further comprises from 0.1 to 5 wt % of a suspending agent for the coated particles. Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives. Polyacrylic acid is available commercially as Carbopol 420, Carbopol 488 or Carbopol 493. Polymers of acrylic acid cross-linked with a polyfunctional agent may also be used, they are available commercially as Carbopol 910, Carbopol 934, Carbopol 940, Carbopol 941 and Carbopol 980. An example of a suitable copolymer of a carboxylic acid containing a monomer and acrylic acid esters is Carbopol 1342. All Carbopol (trade mark) materials are available from Goodrich.

Suitable cross-linked polymers of acrylic acid and acrylate esters are Pemulen TR1 or Pemulen TR2. A suitable heteropolysaccharide gum is xanthan gum, for example that available as Kelzan mu.

The suspending agent is preferably a polymeric suspending agent.

### Styling Polymers

If the product is a styling product it is preferred if a styling polymer is present

The hair styling polymer if present is preferably present in the compositions of the invention in an amount of from 0.001% to 10% by weight, more preferably from 0.1% to 10% by weight, such as from 1% to 8% by weight.

Hair styling polymers are well known. Suitable hair styling polymers include commercially available polymers that contain moieties that render the polymers cationic, anionic, amphoteric or nonionic in nature. Suitable hair styling polymers include, for example, block and graft copolymers. The polymers may be synthetic or naturally derived.

The amount of the polymer may range from 0.5 to 10%, preferably 0.75 to 6% by weight based on total weight of the composition.

Examples of anionic hair styling polymers are:

copolymers of vinyl acetate and crotonic acid;
terpolymers of vinyl acetate, crotonic acid and a vinyl ester of an alpha-branched saturated aliphatic monocarboxylic acid such as vinyl neodecanoate;
copolymers of methyl vinyl ether and maleic anhydride (molar ratio about 1:1) wherein such copolymers are 50% esterified with a saturated alcohol containing from 1 to 4 carbon atoms such as ethanol or butanol;
acrylic copolymers containing acrylic acid or methacrylic acid as the anionic radical-containing moiety with other monomers such as: esters of acrylic or methacrylic acid with one or more saturated alcohols having from 1 to 22 carbon atoms (such as methyl methacrylate, ethyl acrylate, ethyl methacrylate, n-butyl acrylate, t-butyl acrylate, t-butyl methacrylate, n-butyl methacrylate, n-hexyl acrylate, n-octyl acrylate, lauryl methacrylate and behenyl acrylate); glycols having from 1 to 6 carbon atoms (such as hydroxypropyl methacrylate and hydroxyethyl acrylate); styrene; vinyl caprolactam; vinyl acetate; acrylamide; alkyl acrylamides and methacrylamides having 1 to 8 carbon atoms in the alkyl group (such as methacrylamide, t-butyl acrylamide and n-octyl acrylamide); and other compatible unsaturated monomers.

The polymer may also contain grafted silicone, such as polydimethylsiloxane.

Specific examples of suitable anionic hair styling polymers are:

RESYN® 28-2930 available from National Starch (vinyl acetate/crotonic acid/vinyl neodecanoate copolymer);
ULTRAHOLD® 8 available from BASF (CTFA designation Acrylates/acrylamide copolymer);
the GANTREZ®ES series available from ISP corporation esterified copolymers of methyl vinyl ether and maleic anhydride).

Other suitable anionic hair styling polymers include carboxylated polyurethanes. Carboxylated polyurethane resins are linear, hydroxyl-terminated copolymers having pendant carboxyl groups. They may be ethoxylated and/or propoxylated at least at one terminal end. The carboxyl group can be a carboxylic acid group or an ester group, wherein the alkyl moiety of the ester group contains one to three carbon atoms.

The carboxylated polyurethane resin can also be a copolymer of polyvinylpyrrolidone and a polyurethane, having a CTFA designation PVP/polycarbamyl polyglycol ester. Suitable carboxylated polyurethane resins are disclosed in EP-A-0619111 and U.S. Pat. No. 5,000,955. Other suitable hydrophilic polyurethanes are disclosed in U.S. Pat. Nos. 3,822,238; 4,156,066; 4,156,067; 4,255,550; and 4,743,673.

Amphoteric hair styling polymers which can contain cationic groups derived from monomers such as t-butyl aminoethyl methacrylate as well as carboxyl groups derived from monomers such as acrylic acid or methacrylic acid can also be used in the present invention. One specific example of an amphoteric hair styling polymer is Amphomer®

(Octylacrylamide/acrylates/butylaminoethyl methacrylate copolymer) sold by the National Starch and Chemical Corporation.

Examples of nonionic hair styling polymers are homopolymers of N-vinylpyrrolidone and copolymers of N-vinylpyrrolidone with compatible nonionic monomers such as vinyl acetate. Nonionic polymers containing N-vinylpyrrolidone in various weight average molecular weights are available commercially from ISP Corporation—specific examples of such materials are homopolymers of N-vinylpyrrolidone having an average molecular weight of about 630,000 sold under the name PVP K-90 and are homopolymers of N-vinylpyrrolidone having an average molecular weight of about 1,000,000 sold under the name of PVP K-120.

Other suitable nonionic hair styling polymers are cross-linked silicone resins or gums. Specific examples include rigid silicone polymers such as those described in EP-A-0240350 and cross-linked silicone gums such as those described in WO 96/31188.

Examples of cationic hair styling polymers are copolymers of amino-functional acrylate monomers such as lower alkyl aminoalkyl acrylate, or methacrylate monomers such as dimethylaminoethyl methacrylate, with compatible monomers such as N-vinylpyrrolidone, vinyl caprolactam, alkyl methacrylates (such as methyl methacrylate and ethyl methacrylate) and alkyl acrylates (such as ethyl acrylate and n-butyl acrylate).

Specific examples of suitable cationic polymers are:

copolymers of N-vinylpyrrolidone and dimethylaminoethyl methacrylate, available from ISP Corporation as Copolymer 845, Copolymer 937 and Copolymer 958;
copolymers of N-vinylpyrrolidone and dimethylaminopropylacrylamide or methacrylamide, available from ISP Corporation as Styleze® CC10;
copolymers of N-vinylpyrrolidine and dimethylaminoethyl methacrylate;
copolymers of vinylcaprolactam, N-vinylpyrrolidone and dimethylaminoethylmethacrylate;
Polyquaternium-4 (a copolymer of diallyldimonium chloride and hydroxyethylcellulose);
Polyquaternium-11 (formed by the reaction of diethyl sulphate and a copolymer of vinyl pyrrolidone and dimethyl aminoethylmethacrylate), available from ISP as Gafquat® 734, 755 and 755N, and from BASF as Luviquat® PQ11;
Polyquaternium-16 (formed from methylvinylimidazolium chloride and vinylpyrrolidone), available from BASF as Luviquat® FC 370, FC 550, FC 905 and HM-552;
Polyquaternium-46 (prepared by the reaction of vinylcaprolactam and vinylpyrrolidone with methylvinylimidazolium methosulphate), available from BASF as Luviquat®Hold.

Examples of suitable naturally-derived polymers include shellac, alginates, gelatins, pectins, cellulose derivatives and chitosan or salts and derivatives thereof. Commercially available examples include Kytamer® (ex Amerchol) and Amaze® (ex National Starch).

### Adjuvants

The compositions of the present invention may also contain adjuvants suitable for hair care. Generally such ingredients are included individually at a level of up to 2, preferably up to 1 wt % of the total composition.

Suitable hair care adjuvants, include amino acids and ceramides.

The invention will now be further illustrated by the following, non-limiting Examples.

A number illustrates examples of the invention; a letter illustrates Comparative Examples.

All percentages quoted are by weight based on total weight unless otherwise stated.

### EXAMPLES

### Product Manufacture

A base formulation was prepared in the normal manner. In the Examples according to the invention a pre-mix was prepared by pre-dispersing hydrophobic oil (silicone or cationic oil blend) into water. Particulate material (Ludox or alumina) was then added to the dispersion followed by mixing. The resulting pre-mix was added to the base formulation.

The pH and viscosity were adjusted where necessary.

### Evaluation

The following Examples were tested using the Protocol for Instron Pullthrough Method

The Instron pullthrough technique was developed as a method of measuring tactile attributes relating to “body”.

2 g/10″ hair switches were used. Five are used per product and five pull-throughs are recorded per switch. Each set of five switches is treated with test product together, rinsed and dried.

A single switch was hung from the clamp so that the aperture was in line with the first half an inch. The aperture was reduced to 7.5 mm and the position adjusted so that the switch hung in the centre. The force cell is zeroed and the test started. The switch was pulled upwards and the forces recorded by the Instron force cell. The peak value was noted. The aperture was opened, the switch returned to its starting position and the test repeated 4 times on that switch. The test was repeated for all five switches per product tested and the results recorded.

| TRADE NAME | CHEMICAL NAME | EXAMPLE A | EXAMPLE 1 | EXAMPLE B | EXAMPLE 2 |
|---|---|---|---|---|---|
| Texapon N701 | SodiumLaurylEtherSulfate 1-EO | 12.00 | 12.00 | 12.00 | 12.00 |
| Tegobetain CK | Cocoamidopropylbetaine | 1.60 | 1.60 | 1.60 | 1.60 |
| Carbopol 980 | Carboxymethylcellulose | 0.40 | 0.40 | 0.40 | 0.40 |
| Jaguar C13S | Cationic guar polymer | 0.10 | 0.10 | 0.10 | 0.10 |
| Versene 100 (39%) | Tetrasodium EDTA | 0.078 | 0.078 | 0.078 | 0.078 |
| Sodium Chloride | Sodium Chloride | 1.00 | 1.00 | 1.00 | 1.00 |
| Silicone DC1787 | Crosslinked polydimethylsiloxane | — | — | 1.50 | 1.50 |
| PP18500 Quat/oil blend | Cationic oil blend | 3.00 | 3.00 | — | — |
| Ludox W50 | Colloidal silica | — | 4.50 | — | 7.50 |
| Water and minors | Water | To 100% | To 100% | To 100% | To 100% |

### PP18500 Quat/Oil is:

2.2 wt % Tetranyl AO-1 is a commercial ester quaternary surfactant supplied by Kao Corporation Barcelona. They are blends of mono-, di- and tri-oleoylethyl hydroxyethylmonium methosulfate. 6.3 wt % varisoft TA-100 distearyldimonium chloride ex Goldschmidt 32 wt % sunflower oil, emulsifiers and water(mean diameter-2 um)
Ludox W50 colloidal silica mean diameter with Aluminium chloride modified surface-50 nm
Silicone DC1787 mean diameter-500 nm

| Formulation Code | TVM Score (mN) |
|---|---|
| Example A | 56.90 |
| Example 1 | 143.50 |
| Example B | 51.71 |
| Example 2 | 102.65 |

| TRADE NAME | CHEMICAL NAME | EXAMPLE C | EXAMPLE 3 | EXAMPLE 4 | EXAMPLE 5 | EXAMPLE 6 | EXAMPLE 7 |
|---|---|---|---|---|---|---|---|
| Texapon | SodiumLaurylEtherSulfate | 12.00 | 12.00 | 12.00 | 12.00 | 12.00 | 12.00 |
| N701 | 1-EO | | | | | | |
| Tegobetain | Cocoamidopropylbetaine | 1.60 | 1.60 | 1.60 | 1.60 | 1.60 | 1.60 |
| CK | | | | | | | |
| Carbopol | Carboxymethylcellulose | 0.40 | 0.40 | 0.40 | 0.40 | 0.40 | 0.40 |
| 980 | | | | | | | |
| Jaguar C13S | Cationic guar polymer | — | — | — | 0.10 | — | — |
| Sodium | Sodium Chloride | 1.00 | 1.00 | 1.00 | 1.00 | 1.00 | 1.00 |
| Chloride | | | | | | | |
| Silicone | Polydimethylsiloxane | 1.00 | 1.00 | 1.00 | 1.00 | 1.00 | 1.00 |
| DC1310 | | | | | | | |
| Ludox W50 | Colloidal silica | — | 0.15 | 0.30 | 0.30 | 0.50 | 1.00 |
| | Water and minors | To 100% | To 100% | To 100% | To 100% | To 100% | To 100% |

Silicone DC1310 has a mean diameter of 10 um
Ludox W50 colloidal silica has a mean diameter of 50 nm

| Formulation Code | TVM Score (mN) |
|---|---|
| Example C | 40.39 |
| Example 3 | 64.28 |
| Example 4 | 52.84 |
| Example 5 | 80.97 |
| Example 6 | 67.75 |
| Example 7 | 78.55 |

| Trade Name | Chemical Name | Example 8 | Example 9 |
|---|---|---|---|
| Texapon N701 | SodiumLaurylEtherSulfate | 12.00 | 12.00 |
| | 1-EO | | |
| Tegobetain CK | Cocoamidopropylbetaine | 1.60 | 1.60 |
| Carbopol 980 | Carboxymethylcellulose | 0.40 | 0.40 |
| Jaguar C13S | Cationic guar polymer | — | 0.10 |
| Sodium | Sodium Chloride | 1.00 | 1.00 |
| Chloride | | | |
| Silicone | Polydimethylsiloxane | 1.00 | 1.00 |
| DC1310 | | | |
| Dispal 23N4- | Aluminium Oxide | 0.20 | 0.20 |
| 80 | | | |
| | Water and minors | To 100% | To 100% |

Dispal 23N4-80 colloidal Alumina mean diameter 50 nm.

| Formulation Code | TVM Score (mN) |
|---|---|
| Example C | 40.39 |
| Example 8 | 59.04 |
| Example 9 | 68.00 |

### Example 10

| Ingredient | Example 10/Example D |
|---|---|
| SLES 1-EO | 12.00 |
| CAPB | 1.60 |
| Carbopol 980 | 0.40 |
| Jaguar C13S | 0.10 |
| Fragrance | 0.50 |
| Glydant | 0.10 |
| Kathon CG | 0.04 |
| Versene 100 | 0.20 |
| DC1310 | 1.00 |
| Ludox W50 | 0.30 |
| Water | To 100% |

Example 10 was prepared according to the method of the invention in that a pre-mix of studded particles (Ludox and DC1310) was prepared. Example D was prepared by mixing the agents of the formulation together without the formation of a pre-mix.

The formulations were used to treat hair by washing the hair, cutting a 0.5 cm section and immersing the section in the formulation.

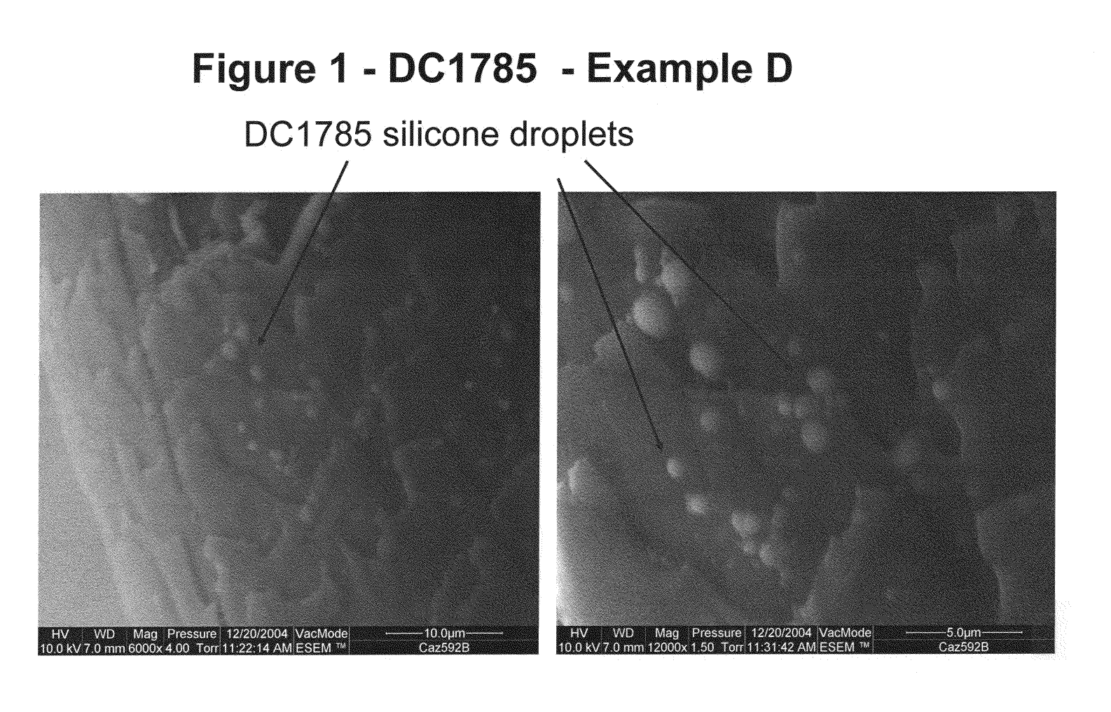

FIG. 1 shows that no studded particles are present in the formulation of Example D, but the studded particles are present in the formulation of Example 10 and are coated onto hair

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A method for enhancing the friction/adhesion between hair fibres by applying a hair treatment composition comprising studded particles, the studded particles comprising liquid hydrophobic oil droplets studded by particulate material, the mean diameter (D3,2) of the studded particles being from 0.01 to 100 microns; the weight ratio of liquid hydrophobic oil droplets to particulate material being from 20:1 to 1:40.

15. (canceled)

16. (canceled)

17. (canceled)

18. A method of treating hair comprising the step of applying to the hair a hair treatment composition comprising an aqueous dispersion of studded particles added to said composition as a pre-mix, the studded particles comprising liquid hydrophobic oil droplets studded by particulate material, the mean diameter (D3,2) of the studded particles being from 0.01 to 100 microns; the weight ratio of liquid hydrophobic oil droplets to particulate material being from 20:1 to 1:40; and wherein said pre-mix is prepared by adding the liquid hydrophobic oil of the studded particle to water to from a dispersion, followed by addition of the particulate material of the studded particle.

19. A method as described in claim 18 wherein the hair treatment composition is applied to style the hair.

20. A method according to claim 18 wherein the hydrophobic oil is an emulsified silicone conditioning oil.

21. A method according to claim 18 in which the particulate material comprises silica, a surface modified silica or mixtures thereof.

22. A method according to claim 18 in which the particulate material has a mean diameter (D3,2) of less than 250 nm.

23. A method according to claim 18 in which within the premix the weight ratio of studded particles to water is 1:1 or less.

24. A method according to claim 20 in which the ratio of silicone hydrophobic oil to particulate material within the pre-mix is from 10:1 to 1:25.

25. A method according to claim 18 in which the pre-mix comprises from 0.01 to 5% by weight of studded particles.

26. A method according to claim 18 in which the pre-mix comprises from 0.01 to 10% by weight of an hydrophobic oil.

27. A method according to claim 18 in which the hair treatment composition further comprises a cationic conditioning surfactant.

28. A method according to claim 18 in which the hair treatment composition further comprises a cleansing surfactant selected from the group consisting of anionic surfactants, non-ionic surfactants, amphoteric surfactants and mixtures thereof.

29. A method according to claim 18 in which the hair treatment composition further comprises a cationic deposition polymer.

30. A method according to claim 18 in which the hair treatment composition is applied to enhance the friction/adhesion between hair fibres.

31. A method according to claim 18 in which the hair treatment composition is applied to increase the volume of hair.
